(19) United States
(12) Patent Application Publication
(10) Pub. No.: US 2009/0112150 A1
(43) Pub. Date: Apr. 30, 2009

(54) CAVITATION ENHANCED TREATMENT THROUGH LOCAL DELIVERY

(75) Inventors: Evan C. Unger, Tucson, AZ (US); Rachel Labell, Coatesville, PA (US); Reena Zutshi, Tucson, AZ (US)

Correspondence Address: DALE F. REGELMAN, QUARLES & BRADY, LLP, ONE SOUTH CHURCH AVENUE AVE, STE. 1700, TUCSON, AZ, 85701-1621, US

(73) Assignee: IMARX THERAPEUTICS, INC., Tucson, AZ (US)

(21) Appl. No.: 11/575,388

(22) PCT Filed: Sep. 15, 2005
(86) PCT No.: PCT/US2005/033172

§ 371 Date: Aug. 11, 2008

Related U.S. Application Data
(60) Provisional application No. 60/610,503, filed on Sep. 15, 2004.

(51) Int. Cl. A61M 31/00

(52) U.S. Cl. 604/22; 604/508

(57) ABSTRACT

A method is disclosed to administer to a patient in need thereof a therapeutically effective amount of one or more therapeutic agents. The method provides a patient comprising a blood vessel, supplies a therapeutic agent comprising a plurality of gas-filled microspheres, and supplies a catheter comprising a proximal end, a distal end, and an infusion length disposed adjacent the distal end, where that infusion length is formed to include an infusion pattern comprising a plurality of apertures extending therethrough. The method catheterizes the blood vessel using the catheter, prepares an aqueous mixture comprising the first therapeutic agent, disposes that aqueous mixture in a container, interconnects the container to the proximal end of said catheter, and administers the aqueous mixture into the blood vessel through the plurality of apertures extending through the catheter.

### CROSS-REFERENCE TO RELATED APPLICATIONS

This application claims priority from a U.S. Provisional Application having Ser. No. 60/610,503 filed Sep. 15, 2004.

### BACKGROUND OF THE INVENTION

It is known in the art to administer therapeutic agents systemically. Using such delivery methods, the agent equilibrates throughout the body in accordance with pharmacological properties. For example, an extracellular fluid agent (“ECF”) will equilibrate throughout the body into the extracellular fluid comprising a volume equal to about 40% of a typical patient's body weight. As an example and assuming a density of about 1 gram/cc, 40 weight percent of a 70 kg patient equals 32 kilograms which corresponds to a fluid volume of about 32 liters.

As those skilled in the art will appreciate, administering one or more therapeutic agents via a patient's ECF results in dispersal of those one or more agents by dilution into the ECF according to the partition coefficients for those one or more agents. On the other hand, if cell specific targeting is employed, an agent may be selectively accumulated by certain target cells. Nonetheless, such target methods are still subject to certain barriers, including without limitation cellular barriers, pressure gradients, and the like.

### SUMMARY OF THE INVENTION

Applicants' invention includes an apparatus and method for improved delivery of one or more therapeutic agents, where that apparatus and method are applicable for treating a variety of diseases using a variety of pharmacological agents. Applicants' method delivers locally a plurality of cavitation nuclei, optionally in combination with one or more additional therapeutic agents. In certain embodiments, the plurality of cavitation nuclei, with or without one or more additional therapeutic agents, are administered using a catheter inserted into a vessel, where that catheter preferably includes a plurality of apertures in the wall portion inserted into the vessel. Applicants' method administers the plurality of cavitation nuclei, with or without additional agents, using that catheter.

### BRIEF DESCRIPTION OF THE DRAWINGS

The invention will be better understood from a reading of the following detailed description taken in conjunction with the drawings in which like reference designators are used to designate like elements, and in which:

FIG. 1 is a block diagram showing a first embodiment of Applicants' infusion apparatus;

FIG. 2 is a block diagram showing a second embodiment of Applicants' infusion apparatus;

FIG. 3 is a block diagram showing a third embodiment of Applicants' infusion apparatus;

FIG. 4 is a cross-sectional view of a polyethylene imine particle comprising a coating of DNA particles.

### DETAILED DESCRIPTION OF THE PREFERRED EMBODIMENTS

This invention is described in preferred embodiments in the following description with reference to the Figures, in which like numbers represent the same or similar elements. Applicants' invention comprises an apparatus and method for improved delivery of one or more therapeutic agents, where that apparatus and method are applicable for treating a variety of diseases using a variety of pharmacological agents. Applicants' method delivers locally a plurality of cavitation nuclei, optionally in combination with one or more additional therapeutic agents. In certain embodiments, the plurality of cavitation nuclei, with or without one or more additional therapeutic agents, are administered using a catheter inserted into a vessel, where that catheter preferably includes a plurality of apertures in the wall portion inserted into the vessel. Applicants' method administers the plurality of cavitation nuclei, with or without additional agents, using that catheter.

By “aperture,” Applicants mean a discontinuity formed in the catheter wall such that a liquid composition disposed within the lumen is released through that discontinuity. In certain embodiments, such apertures comprises holes formed in the catheter wall. In certain embodiments, such apertures comprise slits formed in the catheter wall.

In certain embodiments, Applicants' cavitation nuclei comprise microspheres. By “microsphere,” Applicants mean a material comprising at least one internal void. In certain embodiments, Applicants' microspheres comprise a plurality of phosphorus-containing compounds, such as for example and without limitation dipalmitoylphosphatidylethanolaminepolyethylene glycol (“DPPE-PEG”), dipalmitoylphosphatidylcholine (“DPPC”), and dipalmitoylphosphatidic acid (“DPPA”).

Each of these phosphorus-containing compounds are structurally similar to naturally-occurring lipid/phospholipid materials. As those skilled in the art will appreciate, lipids comprise a polar, i.e. hydrophilic, head and one to three nonpolar, i.e. hydrophobic, tails. Phospholipids comprise materials having a hydrophilic head which includes a positively charged group linked to the tail by a negatively charged phosphate group. described above. Those phosphorus-containing compounds form lipid-like structures in an aqueous medium. References herein to “lipids” refer to any combination of Applicants' plurality of phosphorus-containing compounds.

In any given microsphere, the lipids may be in the form of a monolayer or bilayer, and the mono- or bilayer lipids may be used to form one or more mono- or bilayers. In the case of more than one mono- or bilayer, the mono- or bilayers are generally concentric. The microspheres described herein include such entities commonly referred to as liposomes, micelles, bubbles, microbubbles, vesicles, and the like. Thus, the lipids may be used to form a unilamellar microsphere (comprised of one monolayer or bilayer), an oligolamellar microsphere (comprised of about two or about three monolayers or bilayers) or a multilamellar microsphere (comprised of more than about three monolayers or bilayers). The internal void of the microsphere is filled with a fluorine-containing gas; a perfluorocarbon gas, more preferably perfluoropropane or perfluorobutane; a hydrofluorocarbon gas; or sulfur hexafluoride; and may further contain a solid or liquid material, including, for example, a targeting ligand and/or a bioactive agent, as desired.

Applicants' method selectively delivers a plurality of gas-filled microspheres, i.e. microbubbles, to a treatment site. The microbubbles preferably have a mean diameter less than about 2-3 microns in size. Applicants' method further utilizes the cavitational effects of ultrasound energy. This method takes advantage of the tendency of Applicants' microbubbles to act as cavitation nuclei. The cavitational mechanisms of ultrasonic therapy can be accentuated by the presence of Applicants' microbubbles.

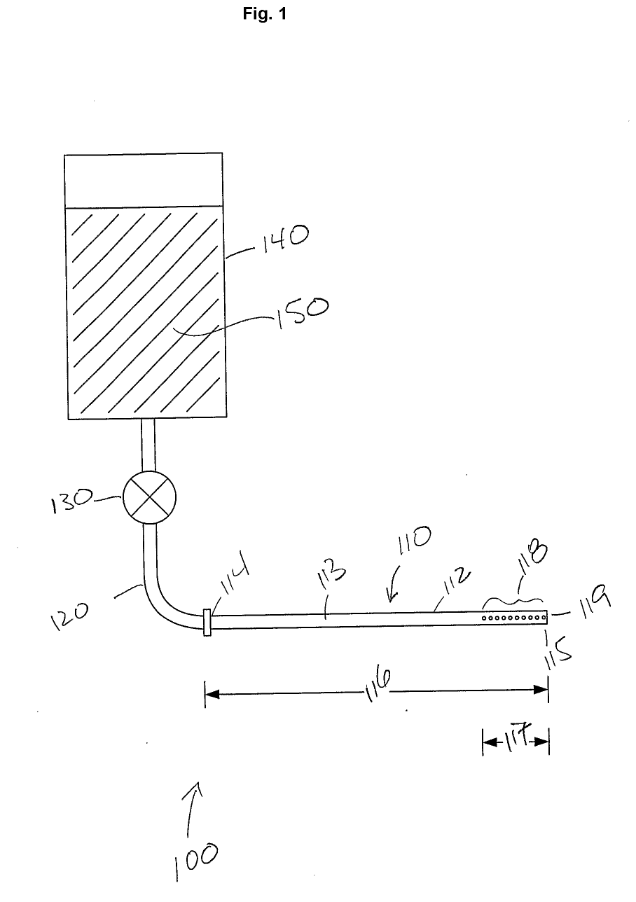

Referring now to FIG. 1, in certain embodiments of Applicants' method a plurality of cavitation nuclei in combination with an aqueous-based pharmaceutically acceptable carrier, and in optional combination with one or more additional therapeutic agents, are infused using apparatus 100. Apparatus 100 includes catheter 110, adset 120, flow rate adjustment mechanism 130, reservoir 140, and fluid 150.

In certain embodiments of Applicants' apparatus and method, fluid 150 comprises Applicants' plurality of cavitation nuclei in combination with an aqueous-based pharmaceutically acceptable carrier, and in optional combination with one or more additional therapeutic agents.

Reservoir 140 and adset 120 are interconnected via flow rate adjustment mechanism 130. Flow rate adjustment mechanism 130 regulates the rate at which fluid 150 is introduced into catheter 110. In certain embodiments, flow rate adjustment mechanism 130 comprises a valve which is adjusted manually. In these embodiments, the level of fluid 150 is maintained at a greater gravitational potential than end 115 of catheter 110. In these embodiments, fluid rate adjustment mechanism 130 does not comprise a mechanical pump.

In other embodiments, flow rate adjustment mechanism 130 comprises a pump, where that pump regulates the flow of fluid 150 from reservoir 140 into catheter 110. As those skilled in the art will appreciate, such a pump mechanism includes other elements not shown in FIG. 1, where those elements include, for example, a power source, circuitry, control knobs, and the like. In these pump embodiments, reservoir 140 need not be disposed at a greater gravitational potential than end 115.

Adset 120 interconnects flow rate adjustment mechanism 130 and catheter 110. Adset 120 is selected from various such devices sold in commerce such that adset 120 is compatible with fluid 150.

As those skilled in the art will appreciate, catheter 110 comprises a tubular structure which includes a contiguous wall 112 having an essentially circular or ovoid cross-section where that contiguous wall defines an interior lumen 113. In certain embodiments, catheter 110 is formed from a silicone elastomer.

Catheter 110 further includes proximal open end 114 and distal end 115. In certain embodiments, distal end 115 comprises an open end. In other embodiments, catheter 110 includes end cap 119 disposed over distal end 115 such that the distal end is closed. In certain embodiments, end cap 119 is integrally formed with catheter wall 112.

Catheter 110 has a length 116. In certain embodiments, length 116 is between about 0.05 meters and about 2.5 meters. In certain embodiments, length 116 is about 1.52 meters, i.e. about 5 feet. Catheter 110 has a diameter between about 2 French and about 8 French, preferably 5-6 French. Catheter 114 includes infusion length 117. Infusion length 117 is between about 5 cm and about 200 cm in length. In certain embodiments, infusion length 117 is about 20 cm in length. Catheter 110 further includes an infusion pattern 118 comprising (N) apertures formed within infusion length 117, where each of those (N) apertures extends through the wall 112 of the catheter such that a liquid composition disposed within catheter lumen 113 is released through those (N) apertures. Infusion length 117 is disposed adjacent to distal end 115.

In the illustrated embodiment of FIG. 1, catheter 110 is formed to include a linear infusion pattern which includes 10 apertures. By “linear infusion pattern,” Applicants mean that the apertures comprising that infusion pattern extend through wall 112 along an infusion line where that infusion line is substantially parallel to an axis defined by the center of lumen 113. In certain embodiments, catheter 110 is formed to includes (N) apertures, where those (N) apertures are randomly arranged within infusion length 117. In certain embodiments, catheter 110 is formed to includes (N) apertures, where those (N) apertures are arranged in a spiral pattern within infusion length 117.

In certain embodiments, lumen 110 is formed to include (P) linear infusion patterns within the infusion length. In certain embodiments, each of the (P) infusion patterns includes the same number of apertures. In other embodiments, one or more of the (P) infusion patterns include differing numbers of apertures. For example, certain Mewissen catheters include a 5 cm infusion length formed to includes 10 holes where those 10 the holes define 4 infusion patterns 4 sides of the catheter. Two of those infusion patterns includes 3 holes, and the other two infusion patterns include 2 holes.

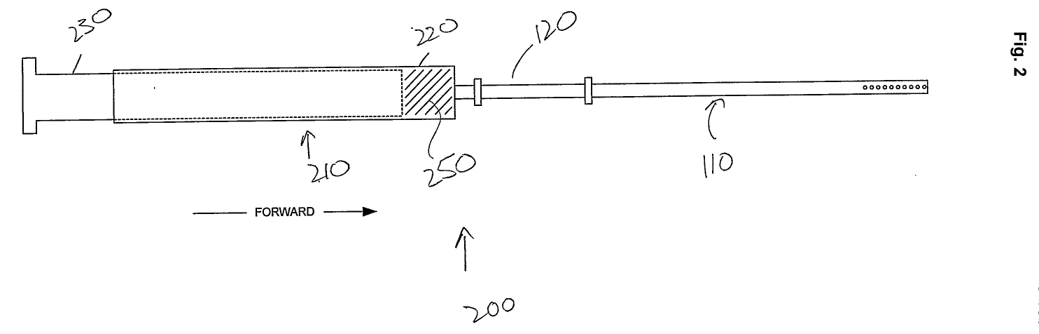

Referring now to FIG. 2, in certain embodiments of Applicants' method a plurality of cavitation nuclei in combination with an aqueous-based pharmaceutically acceptable carrier, and in combination with one or more additional therapeutic agents, are infused using apparatus 200. Apparatus 200 includes catheter 110, adset 120, syringe 210, and fluid 250. In certain embodiments, fluid 250 comprises Applicants' cavitation nuclei composition. In certain embodiments, fluid 150 and fluid 250 are the same. In other embodiments, fluid 150 and fluid 250 differ.

Catheter 110 and adset 120 are described above. As those skilled in the art will appreciate, syringe 210 includes barrel 220 and plunger 230. Fluid 250 is disposed within that portion of barrel 220 not occupied by plunger 230. As those skilled in the art will further appreciate, fluid 250 is introduced into catheter 110 by moving plunger 230 in the forward direction illustrated in FIG. 2. In certain embodiments of Applicants' method, the delivery of fluid 250 from syringe 210 into catheter 110 via adset 120 is performed manually.

In other embodiments, a plurality of cavitation nuclei in combination with an aqueous-based pharmaceutically acceptable carrier, and in combination with one or more additional therapeutic agents, are infused using apparatus 300. Apparatus 300 includes catheter 110, adset 120, syringe 210, fluid 250, in combination with actuator 320 and controller 340. In certain embodiments, syringe 210 and actuator 320 are disposed within housing 310. As those skilled in the art will appreciate, syringe 210, actuator 320, and housing 310, are sometimes referred to as a “syringe pump.” In certain embodiments, controller 340 is internal to housing 310. In other embodiments, controller 340 is external to housing 310. In still other embodiments, controller 340 is remotely located from housing 310.

In external/remote controller embodiments, controller 340 communicates with actuator 320 via communication link 330. In certain embodiments, communication link 330 Communication link 330 is selected from the group comprising a wireless communication link, a serial interconnection, such as RS-232 or RS-422, an ethernet interconnection, a SCSI interconnection, an iSCSI interconnection, a Gigabit Ethernet interconnection, a Bluetooth interconnection, a Fibre Channel interconnection, an ESCON interconnection, a FICON interconnection, a Local Area Network (LAN), a private Wide Area Network (WAN), a public wide area network, Storage Area Network (SAN), Transmission Control Protocol/Internet Protocol (TCP/IP), the Internet, and combinations thereof.

In certain embodiments, communication link 330 is compliant with one or more of the embodiments of IEEE Specification 802.11 (collectively the “IEEE Specification”). As those skilled in the art will appreciate, the IEEE Specification comprises a family of specifications developed by the IEEE for wireless LAN technology.

In certain embodiments, controller 340 comprises a processor and microcode, where the processor uses that microcode to operate apparatus 300. In other embodiments, controller 340 comprises a computing device which includes, inter alia, an operating system, one or more processors, and one or more applications, to operate apparatus 300.

The following examples are merely illustrative of the present invention and should not be considered limiting of the scope of the invention in any way. These examples and equivalents thereof will become more apparent to those skilled in the art in light of the present disclosure and the accompanying claims.

### Example 1A

### Delivery of Microbubbles Through a Catheter at a Flow Rate of 1.7 mL/min

A vial of MRX815H microbubbles (ImaRx Therapeutics, Inc., Tucson, Ariz.) was activated by vigorous agitation and allowed to sit for 15 minutes. The vial was gently inverted ten times to ensure a homogenous suspension. About 1.4 mL of the contents of the vial were removed via a syringe and needle, and were injected into a 50 mL saline bag. The bag was inverted ten times to ensure proper mixing. A nitro I.V. adset (Medical Product Specialists, Brea, Calif.) was attached to the bag and the bag was hung on a pole. The adset was attached to a Mewissen catheter (Boston Scientific, Watertown, Mass.) where that catheter included a 5 cm infusion length having 10 apertures disposed therein. The microbubbles were infused at a rate of 1.7 mL/min. The effluent was analyzed for particle size and total number of particles on an Accusizer 770 (Particle Sizing Systems, Santa Barbara, Calif.) with a 0.5 μM cutoff. Each data point is an average of 3 experiments. The number mean is the average size of the total particles without any mathematical weighting based on volume.

Table I summarizes those measured particle sizes and number of particles. Each data point in Table I is an average of 3 experiments.

TABLE I

| Formulation code | Total # particles/mL | Num. mean | Vol. Mean |
|---|---|---|---|
| 815H-0 vial | 2.08E+10 | 1.02 | 5.28 |
| 815H-0 min | 2.85E+09 | 1.06 | 5.35 |
| 815H-5 min | 4.23E+08 | 0.99 | 4.54 |
| 815H-15 min | 4.16E+08 | 0.98 | 4.45 |
| 815H-25 min | 5.50E+08 | 0.97 | 4.35 |

The data for Formulation Code 815H-0 vial represents data from a sample obtained from the vial sold in commerce. The Formulation Code designations 815H-X min represents data obtained for the effluent, i.e. the fluid released from the infusion length of the catheter at the X minute.

### Example 1B

### Delivery of Microbubbles Through a Catheter at a Flow Rate of 0.3 mL/min

About 2.8 mL of the activated product (MRX815H) was diluted into 17.2 mL of saline in a 20 mL syringe. The syringe was loaded on a syringe pump (Sage Instruments, Boston, Mass.) and connected through a 5-ft tubing to the catheter. The tubing was primed and the diluted product was infused slowly at a flow rate of about 0.3 mL/min. After the completion of infusion, the connector tubing containing the diluted product (volume corresponding to the dead volume of the tubing) was flushed with saline (in a 20 mL syringe) using the syringe pump set at the same flow rate (0.3 mL/min). The fluid released from the catheter was analyzed for size and number of particles on an Accusizer 770 (Particle Sizing Systems, Santa Barbara, Calif.) with a 0.5 μM cutoff. Table II summarizes the results.

TABLE II

| Formulation code | Total # particles/mL | Num. mean | Vol. Mean |
|---|---|---|---|
| 815H-0 vial | 9.55E+09 | 1.08 | 12.71 |
| 815H-0 min | 3.82E+09 | 1.08 | 4.45 |
| 815H-15 min | 2.44E+09 | 0.92 | 1.70 |
| 815H-25 min | 2.02E+09 | 0.91 | 1.85 |
| 815H-35 min | 1.58E+09 | 0.81 | 3.05 |
| 815H-45 min | 1.32E+09 | 0.79 | 3.51 |
| 815h-55 min | 8.72e+08 | 0.79 | 8.45 |

### Example 2

### Loading of Thrombolytic Drugs into the Microbubbles

This experiment demonstrates that administering thrombolytic drugs in combination with microbubbles does not affect the physical properties of those microbubbles. MX115 was activated in the vial. Different concentrations of thrombolytic drugs, Streptokinase (Sigma, Milwaukee, Wis.) and t-PA (Genentech, South San Francisco, Calif.), were added to the vial and incubated with the microbubbles for 5 minutes before analyzing the mixtures using a Model 770 Accusizer (Particle Sizing Systems, Santa Barbara, Calif.). Addition of the drugs did not change the particle size or the particle count significantly. As much as 5 mg of the drug could be loaded into the MRX 115 microbubbles. Table III summarizes the data obtained.

TABLE III

| | Protein | Volume | Number | |
|---|---|---|---|---|
| | loading in | wt | wt | |
| | microbubbles | mean | mean | Number of |
| Protein | (mg) | (μ) | (μ) | particles/mL |
| 0 | 0 | 16.0 | 2.0 | 1.3 × 10⁹ |
| Streptokinase | 0.1 | 13.6 | 1.79 | 1.1 × 10⁹ |
| | 1 | 17.1 | 1.87 | 1.1 × 10⁹ |
| | 5 | 52.9 | 2.44 | 0.5 × 10⁹ |
| tPA | 0.010 | 23.5 | 2.27 | 1.1 × 10⁹ |
| tPA (After 24 hrs) | | 24.4 | 2.20 | 1.3 × 10⁹ |

### Example 3

### Imaging and Cavitation of Microbubbles Delivered at a Flow Rate of 1.7 mL/min

A vial of MRX815H microbubbles was activated and allowed to sit for 15 minutes. The vial was gently inverted ten times to ensure a homogenous suspension. The contents of the vial (1.4 mL) were removed from the vial via a syringe and needle and were injected into a 50 mL saline bag. The bag was inverted ten times to ensure proper mixing. A nitro I.V. adset (Medical Product Specialists, Brea, Calif.) was attached to the bag and the bag was hung on a pole. The adset was attached to a Mewissen catheter (Boston Scientific, Watertown, Mass.) with 5 cm infusion length formed to include 10 apertures. The end of the catheter was threaded through another nitro adset with saline flowing through it and connected to silastic tubing (Dow Corning Corporation, Midland, Mich.).

The end of the catheter was positioned inside the piece of silastic tubing that was acoustically transparent and was suspended in a water bath. The microbubbles were infused at a rate of 1.7 mL/min. The microbubbles released from the catheter and into a pseudo-lumen, and were imaged by suspending a 7.5 MHz PV probe from a diagnostic ultrasound machine (Model 5200S, Acoustic Imaging, Tempe, Ariz.) with low mechanical index (“MI”) into the water directly above the catheter. The microbubbles were visualized streaming out of the apertures in the catheter. A cloud was visualized around the catheter as the microbubbles first filled the lumen of the catheter, and then permeated the space surrounding the catheter.

In order to visualize the destruction of the microbubbles, a therapeutic ultrasonic probe with 10 Watts/cm² and CW (Model V, Richmar Corp., Inola, Okla.) was placed along side the diagnostic probe and angled toward the portion of the catheter being imaged. The application of the therapeutic ultrasound energy effectively destroyed the microbubbles. That destruction was evidenced by the observed loss of contrast. Once the therapeutic ultrasound probe was removed from the water, the microbubbles refilled the lumen and could be again visualized.

### Example 4A

### Simultaneous Delivery of t-Pa and Microbubbles and Subsequent Imaging and Cavitation of the Microbubbles

Activated MRX815H (1.4 mL) is injected into a 50 mL saline bag (Baxter, Deerfield, Ill.). Then 4 mL Tissue Plasminogen Activator (t-PA) comprising a 1 mg/mL solution (Genentech, South San Francisco, Calif.) is injected into the bag. The bag is inverted ten times to ensure proper mixing. A nitro I.V. adset (Medical Product Specialists, Brea, Calif.) is attached to the bag and the bag hung on a pole. The adset is attached to a Mewissen catheter (Boston Scientific, Watertown, Mass.) with 5 cm of side holes (10 total holes). The microbubbles are infused at a rate of 1.7 mL/min. Therefore, the microbubbles are delivered through the adset and released from the catheter.

Imaging is performed with low MI ultrasound. A cloud is visualized around the catheter as the microbubbles first fill the lumen of the catheter, and then permeated the space surrounding the catheter. After optimizing the visualization of cavitation nuclei, the microbubbles are activated with sufficient ultrasonic energy to create radiation force to drive microbubbles into desired tissue, and to activate those microbubbles, i.e. the plurality of cavitation nuclei, to create a local driving force, where that driving force is useful for delivery of the therapeutic agent portion of the infused material.

### Example 4B

### Sequential Delivery of t-Pa and Microbubbles and Subsequent Imaging and Cavitation of the Microbubbles

A vial of MRX815H microbubbles is activated and allowed to sit for 15 min. The vial is gently inverted ten times to ensure a homogenous suspension. The contents of the vial (1.4 mL) are removed from the vial via a syringe and needle and injected into a 50 mL saline bag (Baxter, Deerfield, Ill.). The bag is inverted ten times to ensure proper mixing. A nitro I.V. adset (Medical Product Specialists, Brea, Calif.) is attached to the bag and the bag hung on a pole. The t-PA solution (3-4 mL, Genentech, South San Francisco, Calif.) is loaded into a syringe and attached to the catheter and infused through the catheter with a slow push. Then, the adset is attached to a Mewissen catheter (Boston Scientific, Watertown, Mass.) with 5 cm infusion length having 10 apertures disposed therein. The microbubbles are infused at a rate of 1.7 mL/min. The microbubbles are delivered through the adset and released from the catheter. Imaging is performed with low MI ultrasound. A cloud is visualized around the catheter as the microbubbles first filled the lumen of the catheter and then permeated the space surrounding the catheter. When optimizing visualization of cavitation nuclei, the microbubbles are activated with sufficient ultrasonic energy to create radiation force to drive microbubbles into desired tissue, and to activate those microbubbles, i.e. cavitation nuclei, to create local driving force, where that local force is useful for drug delivery, where that delivered drug has a useful bioeffect.

### Example 5

### Treatment of Acute Limb Ischemia with Microbubbles and Ultrasound

In a feasibility study involving 12 patients, 6 of the 12 patients receive thrombolytic therapy (t-PA) delivered as a bolus of 1 mg/10 cm clot to lace the clot immediately prior to treatment. All patients receive catheter-mediated microbubbles in conjunction with ultrasound. Six patients are treated with ultrasound at 0.8 Watts/cm² (100% duty cycle) and six patients are treated with ultrasound energy at 6.0 Watts/cm² (20% duty cycle). Patients are randomized to t-PA or no t-PA, and to one of the two ultrasound levels. Tables IV and V recite the treatments administered.

TABLE IV

| METHOD OF | NO. OF | | |
|---|---|---|---|
| TREATMENT | PATIENTS | PERFLUTREN LIPID MICROSPHERES | ULTRASOUND |
| Catheter- | 3 | 1.4 cc microbubbles diluted with 8.6 cc | 0.8 W/cm² @ |
| mediated | | normal saline × 2 over 60 minutes (total dose | 100% duty cycle |
| localized | | of microbubbles is 2.8 cc/60 minutes) with 1 mg | |
| Microbubbles + | | tPA/10 cm clot bolus | |
| t-PA bolus | 3 | 1.4 cc microbubbles diluted with 8.6 cc | 6.0 W/cm² @ |
| | | normal saline × 2 over 60 minutes (total dose | 20% duty cycle |
| | | of microbubbles is 2.8 cc over 60 minutes) | |
| | | with 1 mg tPA/10 cm clot bolus | |

TABLE V

| METHOD OF | NO. OF | | |
|---|---|---|---|
| TREATMENT | PATIENTS | PERFLUTREN LIPID MICROSPHERES | ULTRASOUND |
| Catheter- | 3 | 1.4 cc microbubbles diluted with 8.6 cc | 0.8 W/cm² @ |
| mediated | | normal saline × 2 over 60 minutes (total dose | 100% duty cycle |
| localized | | of microbubbles is 2.8 cc/60 minutes) | |
| Microbubbles | 3 | 1.4 cc microbubbles diluted with 8.6 cc | 6.0 W/cm² @ 20% |
| | | normal saline × 2 over 60 minutes (total dose | duty cycle |
| | | of microbubbles is 2.8 cc over 60 minutes) | |

A vascular sheath is placed with standard angiographic technique, generally from catheterizing the opposite femoral artery. The sheath is generally passed across from the contralateral iliac artery and positioned proximal to the level of arterial obstruction. An infusion catheter is then advanced co-axially through the sheath into the thrombus. Diagnostic ultrasound is performed prior to clot lysis to confirm that a satisfactory acoustic window is present to allow transmission of therapeutic ultrasound. During the procedure low mechanical index (“MI”) ultrasound imaging is performed to adjust positioning of the therapeutic transducers and also to optimize application of therapeutic ultrasound with the concentration of microbubbles. The therapeutic ultrasound is applied when sufficient contrast is seen on low MI imaging in the affected segment of the graft.

Patients entering the study are given an IV bolus of Heparin (80-100 U/kg) followed by infusion at a rate of up to 18 U/kg/hr via the arterial sheath. The dose of heparin would be adjusted as per the physician to maintain the ACT at about 2-2.5 times the individual patients control time. ACTs would be acquired prior to treatment and every 30 minutes during treatment until the target anti-coagulation level is achieved.

Prior to commencing the treatment, acoustic transmission gel is liberally applied to the skin. Application of the gel is guided by the marks previously applied to the skin outlining the position of the underlying arteries. Patients who are randomized to the t-PA arm of the study receive 1 mg t-PA for every 10 centimeters of clot as a bolus to lace the clot prior to infusion of microbubbles. Microbubbles are infused at a rate of 2.8 cc/hour for 60 minutes giving a total dose of microbubbles of 2.8 cc/hr.

During this time ultrasound is applied to the overlying skin using ultrasound transducer(s) operating at one (1) megahertz and one of two different power levels. During initial treatment, the transducer is positioned to cover the proximal part of the clot for the first 20 minutes; and then moved to middle third for next 20 minutes, and then again moved to cover the distal third for last 20 minutes. In certain embodiments, during the infusion the catheter is repositioned as necessary so that the infusion side holes were within the region of thrombus under insonation. After 60 minutes of ultrasound treatment, the ultrasound power and microbubble infusion are stopped.

### Example 6

### Treatment of Deep Vein Thrombosis with Microbubbles and Ultrasound

A pilot feasibility study was conducted in 24 patients with DVT. The first 12 patients received catheter-mediated microbubbles without t-PA and the second 12 patients received catheter-mediated microbubbles+t-PA. The dose of t-PA was 5 mg as a bolus to lace the clot and 5 mg administered as an infusion during ultrasound treatment through the catheter (co-administered with the micro bubbles). The first 6 of each group were treated with ultrasound at 0.8 Watts/cm² (100% duty cycle) and the second 6 patients with ultrasound at 6.0 Watts/cm² (20% duty cycle). There was no control group of patients. The study was performed to determine the safety and demonstrate the potential effectiveness of the microbubble product MRX-815 (Perflutren Lipid Microspheres, ImaRx Therapeutics, Inc., Tucson, Ariz.) and clinical ultrasound using the AutoSound (Rich-Mar Corp., Inola, Okla.). The purpose of this pilot study was to determine the feasibility of microbubbles and therapeutic ultrasound for the treatment of patients with acute DVT involving the lower extremities (i.e. popliteal and/or femoral veins; calf vein may be involved). Table VI recites the treatments used.

TABLE VI

| METHOD OF | NO. OF | | |
|---|---|---|---|
| TREATMENT | PATIENTS | MRX-815 | ULTRASOUND |
| Catheter- | 6 | 1.4 cc microbubbles diluted | 0.8 W/cm² @ |
| mediated | | with 50 cc normal saline (51.4 cc | 100% duty cycle |
| localized | | volume after dilution) × 2 | |
| Microbubbles + | | over 60 minutes (total dose of | |
| Intravenous | | microbubbles is 2.8 cc over 60 minutes) | |
| Heparin | | | |
| | 6 | 1.4 cc microbubbles diluted | 6.0 W/cm² @ |
| | | with 50 cc normal saline (51.4 cc | 20% duty cycle |
| | | volume after dilution) × 2 | |
| | | over 60 minutes (total dose of | |
| | | microbubbles is 2.8 cc over 60 | |
| | | minutes) | |
| Intravenous | 6 | 1.4 cc microbubbles diluted | 0.8 W/cm² @ |
| Microbubbles + | | with 50 cc normal saline (51.4 cc | 100% duty cycle |
| Intravenous | | volume after dilution) × 2 | |
| Heparin | | over 60 minutes (total dose of | |
| | | microbubbles is 2.8 cc over 60 | |
| | | minutes) | |
| | 6 | 1.4 cc microbubbles diluted | 6.0 W/cm² @ |
| | | with 50 cc normal saline (51.4 cc | 20% duty cycle |
| | | volume after dilution) × 2 | |
| | | over 60 minutes (total dose of | |
| | | microbubbles 2.8 cc over 60 | |
| | | minutes) | |

Applicants' method which infuses a plurality of cavitation nuclei in combination with an aqueous-based pharmaceutically acceptable carrier, and in combination with one or more additional therapeutic agents, such as for example Heparin, and in combination with therapeutic ultrasound energy includes the following steps. Prior to treatment, patients underwent duplex ultrasound. At the time of ultrasonography, the deep venous system was localized and marked on the overlying skin. This surface marking facilitates positioning of the therapeutic ultrasound transducers. A felt pen or other suitable marker that would not wash away when ultrasound gel is applied to the skin was used to mark the veins.

The appropriate vein was catheterized (inner diameter 4 or 5 Fr., multiple side hole infusion catheter, e.g. Mewissen catheter). Heparin (80-100 U/kg) was injected IV as a bolus and followed by infusion at a rate of up to 18 U/kg/hr. As those skilled in the art will appreciate, a bolus is not required for patients already on heparin therapy. The dose of heparin was adjusted as per the physician to maintain the appropriate anti-coagulation level at about 2-2.5 times the individual patient's control time. Anti-coagulation levels were acquired prior to treatment, and every 30 minutes during treatment until the target anti-coagulation was achieved.

Prior to commencing ultrasound treatment, acoustic transmission gel was liberally applied to the skin. Application of the gel was guided by the markings previously applied to the skin outlining the position of the deep veins. Microbubbles were infused at a rate of 1.7 cc/minute for 60 minutes for a total dose of 2.8 mL microbubbles, during which time ultrasound was applied to the overlying skin using ultrasound transducer(s) operating at about one (1) megahertz and one of two different power levels.

During the initial treatment, the transducer was positioned to cover the proximal third of the clot for the first 20 minutes, the catheter and the ultrasound transducer were then moved to the middle third for next 20 minutes, and the catheter and the ultrasound transducer were then moved to the distal third for last 20 minutes. The treatment time was limited to 60 minutes. After 60 minutes of ultrasound treatment, the ultrasound power and microbubble infusion was stopped. A repeat ultrasound was obtained as soon as practical, but no longer than 60 minutes after the 60-minute period of ultrasound treatment has ended. Additionally the investigators were strongly encouraged to obtain venograms pre and post ultrasound treatment.

### Example 7

The protocol as outlined in Example 6 is performed in a patient with acute myelogenous leukemia who presents with acute DVT involving the calf, popliteal and femoral veins. The infusion is performed using a Mewissen catheter and the patient receives ultrasound treatment without t-PA. The pre ultrasound treatment venograms shows extensive clot involving 30 cm of the venous system with areas of occlusion of greater than 90%. The post ultrasound treatment venograms, performed immediately post ultrasound treatment, shows significant improvement, with about 50% of the venous lumen patent.

### Example 8

A patient with DVT was treated with same protocol as in example 7 (no t-PA). The pre treatment venograms showed occlusion of the superficial femoral vein with filling of superficial venous collaterals. The post ultrasound treatment venograms showed patency of the superficial femoral vein with good flow.

Directions for the correct use of the ultrasound for the ultrasound treatment are below:

### Example 9

### Preparation of Cationic Nanodroplets for Loading of Genetic Material

The formulation of FluoroGene consisted of two steps, the compounding of the lipids into suspension followed by the formation of the nanoparticles with perfluorohexane. FluoroGene has a lipid ratio of 2:1 1,2-dioleoyl-trimethylammonium-propane (DOTAP): L-α-dioleoyl phosphatidylethanolamine (DOPE) with an additional 5% 1,2-dioleoyl-SN-glycero-3-phosphoethanolamine-N-[methoxy(polyethylene glycol)-2000] (mPEG2000 PE). A beaker of saline (300 mL) was heated to 50° C. The DOPE (100 mg, Avanti Polar Lipids, Alabaster, Ala.) was added followed by DOTAP (200 mg, Avanti Polar Lipids, Alabaster, Ala.) and lastly mPEG2000 PE (15 mg, Avanti Polar Lipids, Alabaster, Ala.) and the suspension was stirred for 2 hours. The suspension was homogenized on a Silverson L4RT with a 1 inch tubular mixing unit with a square-hole high shear screen (Silverson Machines LTD, East Longfellow, Minn.) homogenizer at 7500 rpm for 10 minutes. After homogenization the suspension was translucent and homogenous. The lipid suspension was QS to 300 mL and stored in the refrigerator before next step. The cold suspension was put in an ice bath and homogenized on a Silverson at 7500 rpm during a dropwise addition of cold perfluorohexane (6 mL, Aldrich, Milwaukee, Wis.). The suspension was homogenized for 30 min. after addition of perfluorocarbon. Lastly, the suspension was extruded through 47 mm polycarbonate membranes (Whatman, Clifton, N.J.) with 100 nm pore size using an Emulsiflex C5 (Avestin, Ottawa, Ontario). The resulting formulation (1.5 mL) was pipetted into 2 ml glass vials, stoppered, and crimped closed. The formulation was stored at 4° C.

### Example 10

### Delivery of Nanodroplets Containing Genes siRNA and Antisense Oligonucleotides Through Catheter

The FluoroGene formulation of Example 8 was used to bind p-CAT DNA (Lofstrand Labs LTD, Gaithersburg, Md.). A stock solution of p-CAT was prepared with a concentration of 0.5 mg/mL in water. The stock was added to a vial to achieve a 50 μg/mL p-CAT solution (150 μL). The vial was vortexed and allowed to incubate at room temperature for 30 min. Then the DNA loaded FluoroGene was used in in vitro or in vivo experiments.

Another embodiment includes using Fluorogene to deliver siRNA. An example of sense siRNA is the following sequence targeted against Lamin A/C (Elbashir et al, Nature, 2001, 411, 494-498):
Sense siRNA: 5′CUGGACUUCCAGAAGAACAdTdT
Antisense siRNA: 5′ UGUUCUUCUGGAAGUCCAG dTdT

Sense and antisense siRNA are annealed in 100 mM NaCl/50 mM Tris-HCl, pH 8.0 by heating at 94 C for 2 min, cooling to 90° C. for 1 min, then to 20° C. at a rate of 1° C. per minute. The annealed duplex was added to the Fluorogene vial to achieve a final concentration of 200 nM. The vial can then be vortexed and incubated at room temperature for 30 minutes prior to use.

Nanodroplets loaded with genetic material are infused through a catheter as described in Example 1. Such a delivery gives an increased local concentration of the drug loaded nanodroplets which can be then driven into the target cells by application of ultrasound energy.

### Example 11

### Preparation of Nanodroplets for Treating Vulnerable Plaque

Nanodroplets are prepared in two steps which include compounding of the lipids followed by formation of the nanodroplets. Dipalmitoyl phosphatidylserine (DPPS, 20% mole), mPEG5000 PE (4% mole), and DPPC (76% mole) were used for this formulation. Lipids were compounded as described in Example 7 and stored at 4° C. until used for nanoparticle formation.

Nanodroplets were prepared in a Microfluidizer 100 S homogenizer (Microfluidics, Newton, Mass.) with a 30 mL steel chamber. The chamber was cleaned before use by adding de-ionized water up to rim of the chamber. The pump was then engaged to cycle the solution through an 87 μm diamond chamber until the chamber was almost empty. The fluidizer was turned off and filled again and repeated up to 4 times.

After cleaning the chamber, about 30 mLs of cold lipid suspension was added to the chamber, and flushed through the system twice. Then about 29 mLs of cold lipid suspension was added to the chamber. The pump was then engaged to allow recirculation, and the perfluorohexane (Aldrich, Milwaukee, Wis.)/perfluoropentane (Fluoro-Seal, Round Rock, Tex.) mixture (50:50, 600 μL) was added dropwise into the chamber. In addition to perfluorohexane/perfluoropentane (50:50), 1.5 mL of triacetin containing 70 mg/mL paclitaxel was added into the chamber during the initial phase of fluidization. The solution was then fluidized for 20 minutes with a head pressure of 50 psi. After 20 minutes the resulting formulation was opaque. The suspension was removed from the chamber and put into vials, stoppered, and sealed. The nanodroplets were stored at 4° C. until use.

### Example 12

### Preparation of Targeted Nanodroplets for Treatment of Vulnerable Plaque

Nanodroplets capable of targeting and treating vulnerable plaque are prepared in the same manner as in Example 9. The lipids used are formulated to allow the desired targeting. Dipalmitoyl phosphatidylserine (20% mole), mPEG5000 PE (4% mole), DPPC (75% mole), and MRX408 CRGDC-bioconjugate (1% mole) are substituted for the lipids in Example 9.

### Example 13

### Use of Balloon Catheter to Deliver Microbubbles

In this example, a delivery catheter comprising the multi-lumen 6 French Trellis Infusion Catheter (Bacchus Vascular) with two balloons is used, where that catheter is inserted through a thrombotic occlusion or a region of vulnerable plaque and positioned at its distal end with a guide wire. After the distal balloon has been inflated, 4 mg of t-PA (1 mg/mL) are infused followed by inflation of the proximal balloon. The inflated balloons at the proximal and distal end of the occlusion isolate the target area. Once the drug and the microbubbles have been administered to the site, ultrasound energy is then applied to cavitate the bubbles and deliver the thrombolytic drug to the thrombus. In certain embodiments, the drug carrying microbubbles are infused utilizing a syringe pump or a pulsed-spray system capable of intermittently delivering the requisite amount of microbubbles, thereby allowing the bubbles to refresh at the target site before application of ultrasound energy.

### Example 14

### Delivery of Genetic Material Using a Combination of Polyethyeneimine and Microbubbles

Polyethyleneimine, polymer I wherein R1, R2, R3, and R4, are H, particles loaded with genetic material would be prepared by addition of DNA to polyethyleneimine (Sigma, Milwaukee, Wis.) at a molar ratio of 1:10. The weight average molecular weight of the polyethyleneimine is between about 1,000 daltons and about 100,000 daltons.

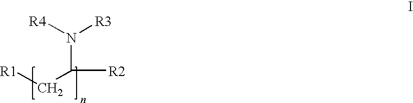

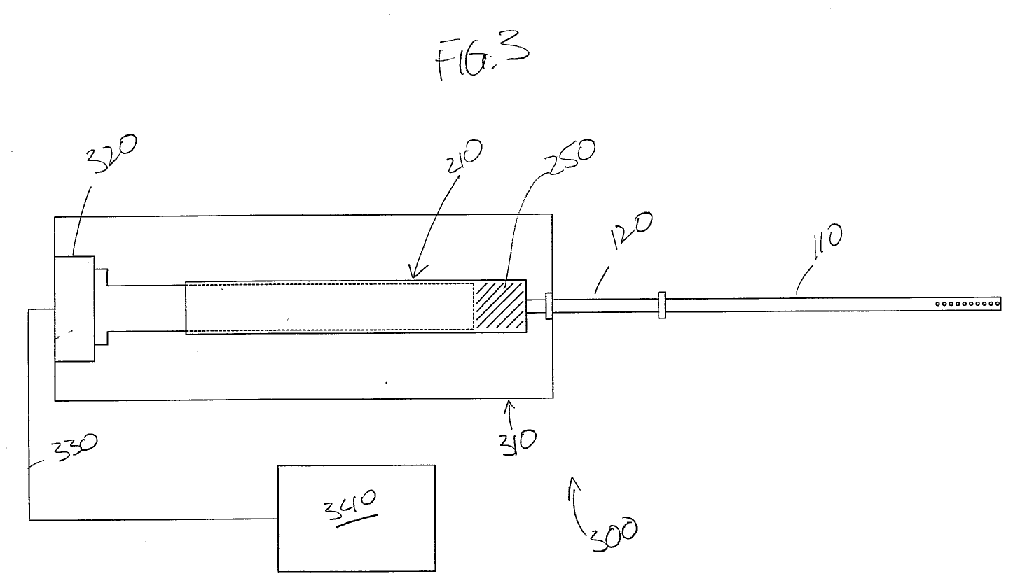

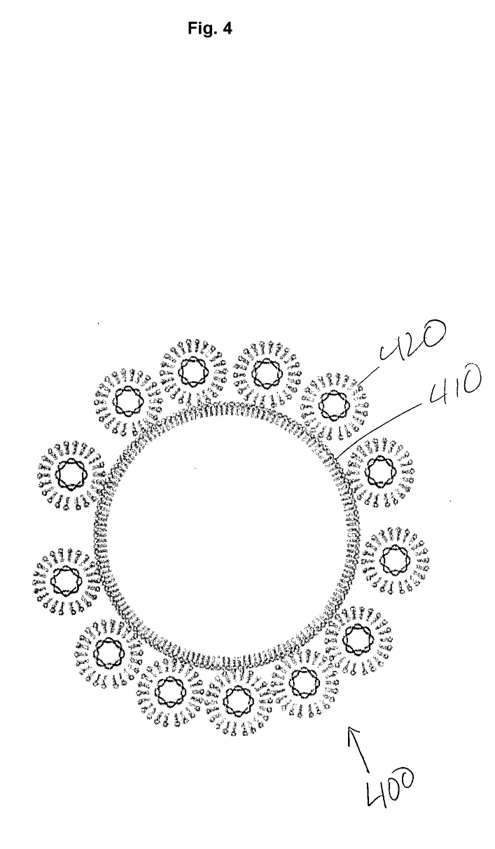

Activated microbubbles are combined with the polyethyleneimine-DNA particles and allowed to incubate. The composition comprises a plurality of acoustically active microbubbles having the outer surface coated with polyethyleneimine-DNA particles. FIG. 4 shows composition 400, which includes microbubble 410 in combination with a plurality of polyethyleneimine-DNA particles 420. Infusion of such a delivery agent through a catheter, such as catheter 110 (FIGS. 1, 2, 3) followed by ultrasound treatment over the target site to cavitate the bubbles could enable the uptake of the genetic material at the target site.

### Example 15

### Infusion of Microbubbles Through Angiodynamics Catheter

The Unifuse multi-side slit catheter made by AngioDynamics is used in place of the catheter mentioned in the following examples; 1A, 1B, 3, 4A, 4B, 5, 6, 7, 10, and Example 14. An experiment was performed to prove the feasibility of using the Unifuse 15 cm treatment length Angiodynamics catheter to deliver MRX815H. Two vials of MRX815H were activated and allowed to sit for 15 min. The vials were inverted ten times to mix and a 3 uL sample was removed for sizing on an AccuSizer 770 (Particle Sizing Systems, Santa Barbara, Calif.) with a 1 μM cutoff. Then, 2.8 mL of the activated product (MRX815H) was diluted into 17.2 mL of saline in a 20 mL syringe. The syringe was loaded on a model 351 syringe pump (Sage Instruments, Boston, Mass.) and connected directly to the catheter.

The diluted product was infused slowly at a flow rate of 0.3 mL/min. At specific time points; t=5, 15, 25, 35, 45, and 55 min., the bubbles coming out from the catheter were analyzed for size and number of particles. Table VII summarizes the results.

TABLE VII

| Formulation code | Total # particles/mL | Num. mean | Vol. Mean |
|---|---|---|---|
| 815H-0 vial | 4.74E+08 | 1.67 | 38.80 |
| 815H-5 min | 1.28E+08 | 1.26 | 11.80 |
| 815H-15 min | 1.19E+08 | 1.16 | 6.47 |
| 815H-25 min | 9.95E+07 | 1.12 | 11.53 |
| 815H-35 min | 7.93E+07 | 1.12 | 17.84 |
| 815H-45 min | 4.40E+07 | 1.17 | 81.33 |
| 815H-55 min | 3.58E+07 | 1.17 | 35.41 |

The side slit design of the AngioDynamics catheter allows a more even distribution, as well as enhanced microbubble release, through the slits when compared to a Mewissen catheter (10 cm, 20 side holes).

### Example 16

### Pulse Spray Using Syringe with Angiodynamics Catheter

The MRX815H microbubbles are prepared and diluted in the same manner as Example 15 only the syringe is loaded into a pulse spray injector instead of a syringe pump. Either the A Mewissen catheter, or a Unifuse catheter, or a Pulse Spray catheter can be used with the pulse spray. The infusion flow rate ranges from 0.1 mL/min to 5 mL/min. An intermittent bolus or pulse is programmed to deliver between 0.1 mL/s to 5 mL/s. The frequency of bolus ranges from every minute to every 30 min. This method of delivery for microbubbles allows maximal filling of the lumen prior to application of ultrasound and thus maximizing the effectiveness of the dissolving process.

### Example 17

### Pulse Spray Microbubbles Using Pulse Spray Pump

Because therapeutic ultrasound destroys the micro bubbles, a pulse spray pump would be synchronized with the ultrasound energy so that the microbubbles would be sprayed out in small doses, e.g. microdoses, substantially less than a milliliter in volume when the ultrasound energy is turned off. After a dose or aliquot of microbubbles is sprayed out from the catheter to enter the target region (e.g. permeate a clot), the ultrasound energy is again activated.

In this example, Applicants' method administers a first portion of the aqueous mixture comprising the microbubbles, and then emits ultrasound energy from the ultrasound emitting device. Thereafter, the method discontinues ultrasound energy emission. Thereafter, the method administers a second portion of the aqueous mixture, and then once again emits ultrasound energy from the ultrasound emitting device.

### Example 18

### Infusion of Microbubbles Through Piezoelectric Catheter, e.g. EKOS, or Other

Another embodiment administers infusions of the micro bubbles via catheter employing an ultrasound equipped catheter. Such a catheter uses a piezoelectric transducer to generate ultrasound energy at the tip of the catheter. Alternatively the piezoelectric elements may be distributed around a guidewire to treat a length of a diseased vessel, e.g. from 1 to 50 cm in length. In another embodiment, photoacoustic stimulation is used to generate the acoustic energy, e.g. the Endovascular Photo Acoustic Recanalization (EPAR) laser system (EndoVasix, Inc, Belmont, Calif.) as described in www.emedicine.com/neuro/topic702.htm.

An example of a piezoelectric catheter is the Ultrasound Thrombolytic Infusion Catheter (EKOS Corporation, Bothell, Wash.), also described in the same reference, which combines the use of a distal ultrasound transducer with infusion of a thrombolytic agent through the microcatheter to disrupt clots. In any case co-administration of the microbubbles improves the rate and effectiveness of the ultrasound treatment. By integrating a pulse-spray, or injection-bolus procedure with application of the ultrasound energy, effectiveness is enhanced. Note that the micro bubbles may be administered intravenously, or proximally be sheath catheter, but local administration is preferred.

### Example 19

### Demonstration of Superiority of Lipid Coated Microbubbles Compared to Albumin Coated Microbubbles

Two samples of microbubbles were compared for their efficiency of catheter delivery, albumin-coated perfluoropropane microbubbles (Optison, Amersham) and MRX-815. The initial concentration of microbubbles was adjusted to the same concentration for the different samples by dilution in saline. The microbubbles were infused through a Mewissen catheter as described above. Approximately 90% of the Optison microbubbles were destroyed by passage through the catheter whereas nearly 100% of the microbubbles from MRX-815 survived transit.

As one skilled in the art would recognize, a wide variety of different microbubble agents may be employed in the above invention including air-filled and PFC gas filled microbubbles. Polymers, synthetic and natural may be used to stabilize the micro bubbles. The microbubbles are preferably less than about 2-3 microns in diameter, and the micro bubbles are preferably coated by lipid.

While the preferred embodiments of the present invention have been illustrated in detail, it should be apparent that modifications and adaptations to those embodiments may occur to one skilled in the art without departing from the scope of the present invention.

We claim:

1. A method to administer to a patient in need thereof a therapeutically effective amount of one or more therapeutic agents, comprising the steps of:
providing a patient comprising a blood vessel, supplying a first therapeutic agent comprising a plurality of gas-filled microspheres;
supplying a catheter comprising a proximal end, distal end, and an infusion length disposed adjacent said distal end, wherein said infusion length is formed to include an infusion pattern comprising a plurality of apertures extending therethrough;
preparing an aqueous mixture comprising said first therapeutic agent;
catheterizing said blood vessel by advancing said distal end of said catheter into said vessel;
disposing said first therapeutic agent in a container;
interconnecting said container to said proximal end of said catheter;
administering said aqueous mixture into said blood vessel through said plurality of apertures.

2. The method of claim 1, wherein said supplying a catheter step further comprising supplying a catheter wherein said infusion pattern comprises a linear infusion pattern.

3. The method of claim 1, wherein said supplying a catheter step further comprising supplying a catheter wherein said infusion pattern comprises a spiral infusion pattern.

4. The method of claim 1, wherein said supplying a catheter step further comprising supplying a catheter wherein said infusion pattern comprises a random infusion pattern.

5. The method of claim 1, further comprising the steps of:
supplying a second therapeutic agent;
wherein said preparing an aqueous mixture step further comprises forming an aqueous mixture comprising said first therapeutic agent and said second therapeutic agent.

6. The method of claim 5, further comprising the steps of:
localizing said blood vessel;
supplying an ultrasound emitting device;
placing said ultrasound emitting device on said patient over said blood vessel;
emitting ultrasound energy from said ultrasound emitting device while administering said aqueous mixture.

7. The method of claim 6, wherein said supplying a catheter step and said supplying an ultrasound emitting device step further comprise supplying a catheter comprising an piezoelectric transducer disposed on said distal end.

8. The method of claim 6, wherein said administering step and said emitting step further comprise the steps of:
administering a first portion of said aqueous mixture;
emitting ultrasound energy from said ultrasound emitting device;
discontinuing ultrasound energy emission;
administering a second portion of said aqueous mixture;
emitting ultrasound energy from said ultrasound emitting device.

9. The method of claim 6, wherein said supplying a second therapeutic agent step comprises:
supplying DNA;
supplying polyethyleneimine;
forming a second therapeutic agent by adding said DNA to said polyethylene imine.

10. The method of claim 6, wherein said supplying a second therapeutic agent step comprises supplying Tissue Plasminogen Activator.

11. The method of claim 10, wherein said emitting ultrasound step further comprises emitting ultrasound energy from said ultrasound emitting device at a power level of 0.8 Watts/cm².

12. The method of claim 10, wherein said emitting ultrasound step further comprises emitting ultrasound energy from said ultrasound emitting device at a power level of 6.0 Watts/cm².

13. The method of claim 1, further comprising the steps of:
supplying a second therapeutic agent;
administering said second therapeutic agent as a bolus before administering said aqueous mixture.

14. The method of claim 13, further comprising the steps of:
localizing said blood vessel;
supplying an ultrasound emitting device;
placing said ultrasound emitting device on said patient over said vein;
emitting ultrasound energy from said ultrasound emitting device while administering said aqueous mixture.

15. The method of claim 14, wherein said supplying a second therapeutic agent step comprises supplying Tissue Plasminogen Activator.

16. The method of claim 14, wherein said supplying a second therapeutic agent step comprises supplying Heparin.

17. The method of claim 14, wherein said emitting ultrasound step further comprises emitting ultrasound energy from said ultrasound emitting device at a power level of 0.8 Watts/cm².

18. The method of claim 14, wherein said emitting ultrasound step further comprises emitting ultrasound energy from said ultrasound emitting device at a power level of 6.0 Watts/cm².

19. A method to treat acute limb ischemia by administering to a patient in need thereof a therapeutically effective amount of Tissue Plasminogen Activator, comprising the steps of:
supplying a plurality of gas-filled microspheres;
supplying Tissue Plasminogen Activator;
supplying an ultrasound emitting device;
supplying a catheter comprising a proximal end, distal end, and an infusion length disposed adjacent said distal end, wherein said infusion length is formed to include an infusion pattern comprising a plurality of apertures extending therethrough;
preparing an aqueous mixture comprising said plurality of gas-filled microspheres;
identifying an artery comprising a clot;
catheterizing said artery by advancing said distal end of said catheter into said artery proximal to said clot;
disposing said aqueous mixture in a container;
interconnecting said container with said proximal end of said catheter;
placing said ultrasound emitting device on said patient over said clot;
administering said Tissue Plasminogen Activator as a bolus through said catheter;
administering said aqueous mixture into said artery through said catheter;
emitting ultrasound energy from said ultrasound emitting device while administering said aqueous mixture.

20. The method of claim 19, wherein:
said clot comprises a proximal portion, a middle portion, and a distal portion;
said placing said ultrasound emitting device step and said emitting ultrasound energy steps further comprise:
placing said ultrasound emitting device over said proximal portion of said clot;
emitting ultrasound energy from said ultrasound emitting device for a first 20 minute time interval;
placing said ultrasound emitting device over said middle portion of said clot;
emitting ultrasound energy from said ultrasound emitting device for a second 20 minute time interval;
placing said ultrasound emitting device over said distal portion of said clot;
emitting ultrasound energy from said ultrasound emitting device for a third 20 minute period.

21. A method to treat deep vein thrombosis by administering to a patient in need thereof a therapeutically effective amount of Tissue Plasminogen Activator, comprising the steps of:
supplying a plurality of gas-filled microspheres;
supplying Tissue Plasminogen Activator;
supplying an ultrasound emitting device;
supplying a catheter comprising a proximal end, distal end, and an infusion length disposed adjacent said distal end, wherein said infusion length is formed to include an infusion pattern comprising a plurality of apertures extending therethrough;
preparing an aqueous mixture comprising said plurality of gas-filled microspheres and a first portion of said Tissue Plasminogen Activator;
identifying a vein comprising an occlusion;
catheterizing said occluded vein by advancing said distal end of said catheter into said vein distal to said occlusion;
disposing said aqueous mixture in a container;
interconnecting said container with said proximal end of said catheter;
placing said ultrasound emitting device on said patient over said occlusion;
administering a second portion of said Tissue Plasminogen Activator as a bolus through said catheter;
administering said aqueous mixture into said vein through said catheter;
emitting ultrasound energy from said ultrasound emitting device while administering said aqueous mixture.
